(19) United States
(12) Patent Application Publication
(10) Pub. No.: US 2026/0283936 A1
(43) Pub. Date: Sep. 24, 2026

(54) Composition For an Organic Skin Moisturizer

(71) Applicant: Sunil Chandra Sutradhar, Miramar, FL (US)

(72) Inventor: Sunil Chandra Sutradhar, Miramar, FL (US)

(21) Appl. No.: 19/572,516

(22) Filed: Mar. 19, 2026

Related U.S. Application Data
(60) Provisional application No. 63/775,084, filed on Mar. 20, 2025.

(51) Int. Cl. A61K 8/92; A61K 8/23; A61K 8/36; A61K 8/365; A61K 8/37; A61K 8/67; A61Q 19/00

(52) U.S. Cl. CPC A61K 8/922; A61K 8/23; A61K 8/361; A61K 8/365; A61K 8/375; A61K 8/678; A61K 8/925; A61Q 19/007; A61K 2800/5922

(57) ABSTRACT

A composition for an organic skincare lotion that works as an effective moisturizer for very dry skin that may have developed due to multiple reasons such as old age, weather conditions, bodywash/facewash, medications, etc. is provided. More specifically, the composition includes naturally sourced ingredients that are skin friendly. Further, the moisturizer enables users to have a protective barrier of natural organic oils, that prevents trans epidermal water loss (TEWL) on skin, such that the skin remains supple with a smooth velvet feel. The composition further uses fresh and sealed ingredients that are non-comedogenic, have specific benefits to the skin and lie within a standard pH range. Furthermore, the ingredients of the composition utilize mild biostatic blends of preservatives that do not interfere with natural skin microbiome or resident flora.

### FIELD OF THE INVENTION

The present invention generally relates to a composition for an organic skin moisturizer. More specifically, the present invention relates to a composition and method of processing a protective barrier of natural organic oils, that prevents trans epidermal water loss (TEWL) on skin, such that the skin remains supple with a smooth velvet feel.

### BACKGROUND OF THE INVENTION

Moisturizers are essential to maintain the skin's hydration, health, and barrier function. In other words, moisturizers are crucial for hydration and protecting the skin barrier, especially for dry, aging, or compromised skin. If used correctly, a moisturizer prevents moisture loss, reduces the appearance of fine lines, manages oil production, and repairs damage from environmental stressors. However, using the wrong type or over-applying a moisturizer can lead to issues like clogged pores, acne, or skin becoming “lazy” in its natural moisturizing processes. Conventional moisturizers use emollients such as petroleum derivatives, silicones, co polyols, animal derived lipids, and other synthetic compounds that are not compatible with the healthy acid mantle of human beings. Further, using oxidized (rancid) oils in an emulsion system reduces the skin's pH below the desired range, thereby compromising the overall quality. Thus, choosing biodegradable ingredients that are non-comedogenic and that help with maintaining the pH level of the user's skin are also important considerations. Additionally, preservatives are used to extend shelf life in most conventional emulsions. These preservatives are biocidal, or they kill all microbes that come in contact with the product. Thus, there is a need for a sustainable, organic moisturizer that uses natural ingredients and natural alternatives, while being skin-friendly.

An objective of the present invention is to formulate a cosmetic emulsion from a natural and neutral standpoint excluding all possible harmful and debatable ingredients. Further, the present invention also aims to fight dry skin issues due to age, disease conditions, side effects of medications or weather change, while providing adequate skin hydration.

### SUMMARY

The present invention is intended to provide users with an organic skincare lotion that works as an effective moisturizer for very dry skin that may have developed due to multiple reasons such as old age, weather conditions, bodywash/facewash, medications, etc. More specifically, the present invention relates to a composition and a method of making thereof, for creating a protective barrier of natural organic oils, that prevents trans epidermal water loss (TEWL) on skin, such that the skin remains supple with a smooth velvet feel. The composition of the present invention uses fresh and sealed ingredients that are natural, organic, non-comedogenic, and have specific benefits to the skin and lie within a standard pH range. Further, the present invention utilizes mild biostatic blends of preservatives that do not interfere with natural skin microbiome or resident flora.

### BRIEF DESCRIPTION OF THE DRAWINGS

FIG. 1 is a block diagram of the present invention illustrating the ingredients of the present invention.

FIG. 2 is a block diagram of the present invention illustrating a generic composition of the present invention.

FIG. 3 is a block diagram illustrating the proportions of the present invention.

FIG. 4 is a continuation of FIG. 3.

### DETAILED DESCRIPTION OF THE INVENTION

All illustrations of the drawings are for the purpose of describing selected versions of the present invention and are not intended to limit the scope of the present invention.

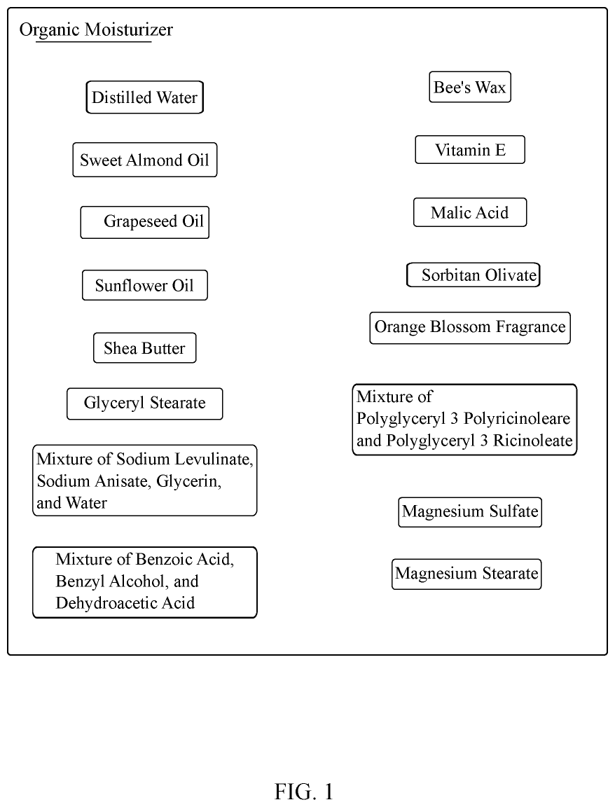

The present invention is a composition for an organic skin moisturizer and a method and making thereof. More specifically, the present invention is an organic skin moisturizer that helps to maintain oil and water balance of dry, cracked or irritated skin, which may have occurred because of body wash/face wash, old age, weather conditions or medications. The present invention is an emulsion that aims at providing adequate skin hydration for users with any of the above conditions through natural ingredients. The following description is in reference to FIG. 1 through FIG. 4.

A mixture of oil and water is called an emulsion. Creams and lotions are non-limiting examples of emulsions. A modern-day comprehensive concept for an emulsion is as follows:

Emulsion=Oil+Interface(Emulsifier(s)+Water+Additives+Preservative(s)+Fragrance.

Except for water, conventional skincare emulsions contain petrochemicals and synthetic compounds. Effects of these chemicals on human skin have long debatable stories that can be found easily online. The present invention isolates all debatable ingredients and includes organic vegetable oils/butter and natural wax instead of synthetic compounds. Thus, the present invention comprises a combination of simple organic oil and moisture providing/retaining components that can treat multiple conditions. The water or moisture element of the present invention helps eliminate dry skin by preventing TEWL. The present invention further neutralizes extra oil for people with oily skin, by creating a protective barrier and supplementing the acid mantle after shower or each face wash. Acid mantle is the invisible coating on human skin surface that is composed of sweat, sebum, dead cell debris and many other components to maintain a slightly acidic barrier to prevent pathogenic microbes and other invaders. Further, the present invention utilizes a mild biostatic blend of preservatives, that does not interfere with the natural skin's microbiome or resident flora.

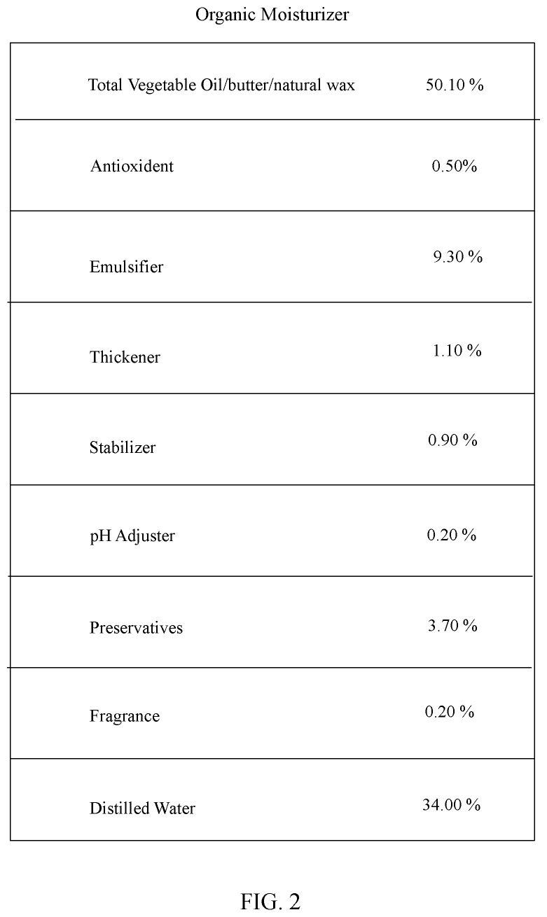

According to a preferred embodiment of the present invention, and as seen in FIG. 2, a generalized composition of the present invention is as follows:

| | |
|---|---|
| Total Vegetable Oil/butter/natural wax | 50.10% |
| Antioxidant | 0.50% |
| Emulsifier | 9.30% |
| Thickener | 1.10% |
| Stabilizer | 0.90% |
| pH Adjuster | 0.20% |
| Preservatives | 3.70% |
| Fragrance | 0.20% |
| Distilled Water | 34.00% |

Vegetable oils are highly inclined to oxidation particularly in presence of water. Oxidized (rancid) oils in the emulsion system reduce pH below the desired range, thus compromising the overall quality. To that end, the present invention uses fresh or sealed ingredients to prevent oxidation. Further, antioxidants and specialized airless containers as well as proper maintenance procedures are carried out, thereby ensuring quality of the product. Furthermore, the present invention has a small batch compounding approach, which helps in retaining the quality of every batch.

According to the preferred embodiment, selection of the oil phase of the composition is done based on the required HLB (hydrophilic-lipophilic balance) values of each ingredient, comedogenic characteristic and feel after application. Emulsifiers are also chosen based on their plant origin and relevant properties to create stable emulsions. The thickener, stabilizer, pH adjuster, etc. are selected based on their natural origins and specific benefits. Preservatives used are mild, biodegradable and suitable to stabilize natural emulsion within a standard pH range. Finally, the fragrance used is a natural essential oil diluted in sunflower oil, for its mild and soothing effect. Thus, according to the preferred embodiment, and as seen in FIG. 1, the formulation of the present invention comprises a quantity of sweet almond oil, a quantity of grapeseed oil, a quantity of sunflower oil, a quantity of shea butter, a quantity of bee's wax, a quantity of vitamin E, a quantity of glyceryl stearate, a quantity of sorbitan olivate, a quantity of a mixture of, polygluceryl 3 polyricinoleare and polyglyceryl 3 ricinoleate, a quantity of magnesium stearate, a quantity of magnesium sulfate, a quantity of malic acid, a quantity of a mixture of, sodium levulinate, sodium anisate, glycerin, and water, a quantity of a mixture of, benzoic acid, benzyl alcohol, and dehydroacetic acid, a quantity of orange blossom fragrance, and a quantity of distilled water, that are homogeneously mixed into an organic moisturizer. Preferably, a method of mixing the ingredients comprises the following step. Components are separately mixed in two phases based on their solubility either in oil or water. Then they are heated up to 80° C., combined and mixed thoroughly with the help of agitators. Subsequently, the mixture is cooled down with slow stirring. Fragrance and other heat sensitive ingredients are added during the cool down phase. However, it should be noted that any other mixing method/techniques using any other components or arrangement of components falls under the scope of the invention, as long as the objectives of the present invention are fulfilled.

As a whole, the water contributes to moisture and the oils provide a barrier. Different oils have a unique set of fatty acids to confer a perfect blend of emollient effect. Emulsifiers act as an interface between oil and water, and preservatives protect the product from spoilage. Further, pH adjusters control the acidity, and fragrance imparts consumers' appeal.

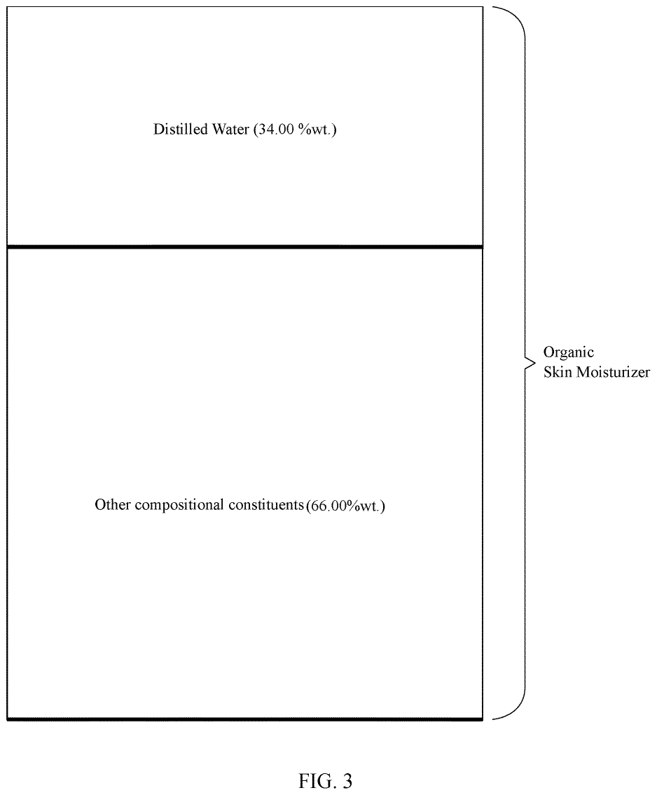

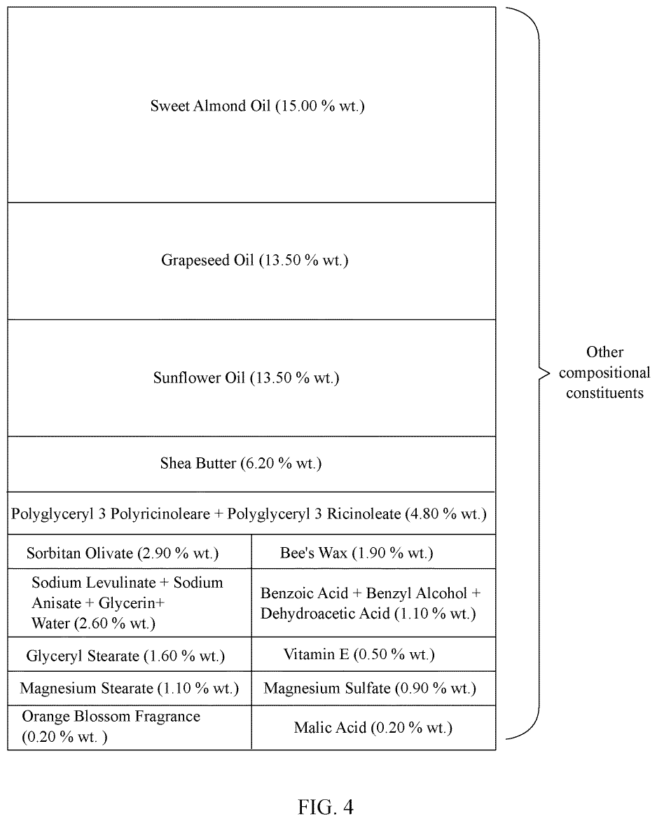

As can be seen in FIGS. 3 and 4, in order to properly proportion the moisturizer, and impart the intended benefits, the quantity of distilled water is approximately 34.00 percent by weight (wt. %) of the organic moisturizer. The other constituent ingredients have the following quantities. The quantity of sweet almond oil is approximately 15.00 wt. % of the organic moisturizer. The quantity of grapeseed oil is approximately 13.50 wt. % of the organic moisturizer. The quantity of sunflower oil is approximately 13.50 wt. % of the organic moisturizer. The quantity of shear butter is approximately 6.20 wt. % of the organic moisturizer. The quantity of bee's wax is approximately 1.90 wt. % of the organic moisturizer. The quantity of vitamin E is approximately 0.50 wt. % of the organic moisturizer. The quantity of glyceryl stearate is approximately 1.60 wt. % of the organic moisturizer. The quantity of sorbitan olivate is approximately 2.90 wt. % of the organic moisturizer. The quantity of the mixture of polygluceryl 3 polyricinoleare and polyglyceryl 3 ricinoleate is approximately 4.80 wt. % of the organic moisturizer. The quantity of magnesium stearate is approximately 1.10 wt. % of the organic moisturizer. The quantity of magnesium sulfate is approximately 0.90 wt. % of the organic moisturizer. The quantity of malic acid is approximately 0.20 wt. % of the organic moisturizer. The quantity of the mixture of sodium levulinate, sodium anisate, glycerin, and water, is approximately 2.60 wt. % of the organic moisturizer. The quantity of the mixture of benzoic acid, benzyl alcohol, and dehydroacetic acid is approximately 1.10 wt. % of the organic moisturizer. The quantity of orange blossom fragrance is approximately 0.20 wt. % of the organic moisturizer. In reference to the aforementioned compositional proportions, the term “approximately” preferably means within an error range of up to +0.005 wt. %.

Thus, the moisturizer of the present invention provides a rich nourishing coat on the skin surface that is not greasy yet provides a silky or velvety feel throughout the day with a baby soft texture.

Although the invention has been explained in relation to its preferred embodiment, it is to be understood that many other possible modifications and variations can be made without departing from the spirit and scope of the invention as hereinafter claimed.

What is claimed is:

1. A composition for an organic hydrating skincare lotion comprising:
a quantity of sweet almond oil;
a quantity of grapeseed oil;
a quantity of sunflower oil;
a quantity of shea butter;
a quantity of bee's wax;
a quantity of vitamin E;
a quantity of glyceryl stearate;
a quantity of sorbitan olivate;
a quantity of a mixture of polygluceryl 3 polyricinoleare and polyglyceryl 3 ricinoleate;
a quantity of magnesium stearate;
a quantity of magnesium sulfate;
a quantity of malic acid;
a quantity of a mixture of sodium levulinate, sodium anisate, glycerin, and water;
a quantity of a mixture of benzoic acid, benzyl alcohol, and dehydroacetic acid;
a quantity of orange blossom fragrance;
a quantity of distilled water; and
the quantity of sweet almond oil, the quantity of grapeseed oil, the quantity of sunflower oil, the quantity of shea butter, the quantity of bee's wax, the quantity of vitamin E, the quantity of glyceryl stearate, the quantity of sorbitan olivate, the quantity of the mixture of polygluceryl 3 polyricinoleare and polyglyceryl 3 ricinoleate, the quantity of magnesium stearate, the quantity of magnesium sulfate, the quantity of malic acid, the quantity of the mixture of sodium levulinate, sodium anisate, glycerin, and water, the quantity of the mixture of benzoic acid, benzyl alcohol, and dehydroacetic acid, the quantity of orange blossom fragrance, and the quantity of distilled water being homogeneously mixed into an organic moisturizer.

2. The composition for the organic hydrating skincare lotion claimed in claim 1 comprises:
the quantity of sweet almond oil being approximately 15.00 percentage by weight (wt. %) of the organic moisturizer.

3. The composition for the organic hydrating skincare lotion as claimed in claim 1 comprises:
the quantity of grapeseed oil being approximately 13.50 wt. % of the organic moisturizer.

4. The composition for the organic hydrating skincare lotion as claimed in claim 1 comprises:
the quantity of sunflower oil being approximately 13.50 wt. % of the organic moisturizer.

5. The composition for the organic hydrating skincare lotion as claimed in claim 1 comprises:
the quantity of shear butter being approximately 6.20 wt. % of the organic moisturizer.

6. The composition for the organic hydrating skincare lotion as claimed in claim 1 comprises:
the quantity of bee's wax being approximately 1.90 wt. % of the organic moisturizer.

7. The composition for the organic hydrating skincare lotion as claimed in claim 1 comprises:
the quantity of vitamin E being approximately 0.50 wt. % of the organic moisturizer.

8. The composition for the organic hydrating skincare lotion as claimed in claim 1 comprises:
the quantity of glyceryl stearate being approximately 1.60 wt. % of the organic moisturizer.

9. The composition for the organic hydrating skincare lotion as claimed in claim 1 comprises:
the quantity of sorbitan olivate being approximately 2.90 wt. % of the organic moisturizer.

10. The composition for the organic hydrating skincare lotion as claimed in claim 1 comprises:
the quantity of the mixture of polygluceryl 3 polyricinoleare and polyglyceryl 3 ricinoleate being approximately 4.80 wt. % of the organic moisturizer.

11. The composition for the organic hydrating skincare lotion as claimed in claim 1 comprises:
the quantity of magnesium stearate being approximately 1.10 wt. % of the organic moisturizer.

12. The composition for the organic hydrating skincare lotion as claimed in claim 1 comprises:
the quantity of magnesium sulfate being approximately 0.90 wt. % of the organic moisturizer.

13. The composition for the organic hydrating skincare lotion as claimed in claim 1 comprises:
the quantity of malic acid being approximately 0.20 wt. % of the organic.

14. The composition for the organic hydrating skincare lotion as claimed in claim 1 comprises:
the quantity of the mixture of sodium levulinate, sodium anisate, glycerin, and water being approximately 2.60 wt. % of the organic moisturizer.

15. The composition for the organic hydrating skincare lotion as claimed in claim 1 comprises:
the quantity of the mixture of benzoic acid, benzyl alcohol, and dehydroacetic acid being approximately 1.10 wt. % of the organic moisturizer.

16. The composition for the organic hydrating skincare lotion as claimed in claim 1 comprises:
the quantity of orange blossom fragrance being approximately 0.20 wt. % of the organic moisturizer.

17. The composition for the organic hydrating skincare lotion as claimed in claim 1 comprises:
the quantity of distilled water being approximately 34.00 wt. % of the organic moisturizer.
